(19) United States
(12) Patent Application Publication
(10) Pub. No.: US 2025/0073118 A1
(43) Pub. Date: Mar. 6, 2025

(54) PHYSIOTHERAPEUTIC APPARATUS RECEPTACLE AND PHYSIOTHERAPEUTIC APPARATUS SYSTEM

(71) Applicant: CHANGSHA ANXIANG MEDICAL TECHNOLOGY CO., LTD., CHANGSHA (CN)

(72) Inventor: JUNYUAN CHEN, CHANGSHA (CN)

(21) Appl. No.: 18/481,216

(22) Filed: Oct. 4, 2023

(30) Foreign Application Priority Data
Aug. 30, 2023 (CN) 202322348202.2

(51) Int. Cl. A61H 23/02

(52) U.S. Cl. CPC A61H 23/02; A61H 2201/0157

(57) ABSTRACT

A physiotherapeutic apparatus receptacle includes: a housing. An accommodation chamber is defined in the housing, wherein a base is arranged at an opening of the accommodation chamber, and the base is recessed towards an interior of the accommodation chamber to define a first accommodation trough and a second accommodation trough. The first accommodation trough and the second accommodation trough are arranged adjacent to each other, and when maintained being connected to each other, a first drive portion and a first patch are simultaneously received in the first accommodation trough and the second accommodation trough respectively.

### CROSS-REFERENCE TO RELATED APPLICATIONS

The present disclosure claims priority to Chinese Patent Application No. 202322348202.2, filed with the Chinese Patent Office on Aug. 30, 2023, titled “PHYSIOTHERAPEUTIC APPARATUS RECEPTACLE AND PHYSIOTHERAPEUTIC APPARATUS SYSTEM”, the entire contents of which are incorporated herein by reference.

### TECHNICAL FIELD

Embodiments of the present disclosure relate to the technical field of physiotherapy, and in particular, relate to a physiotherapeutic apparatus receptacle and a physiotherapeutic apparatus system.

### BACKGROUND

A physiotherapeutic apparatus, that is, physical therapeutic apparatus, applies various physical agents, for example, electricity, light, sound, magnetism and the like, to human bodies, and induces biophysical and biochemical variations to the human body, thereby achieving purposes of prevention and treatment of diseases. The physiotherapeutic apparatus is very welcomed due to good therapy effects on some diseases, convenient use, and the like.

A conventional physiotherapeutic apparatus includes a drive portion, a conductive wire, and a patch. Before use, the drive portion needs to be connected to the patch via the conductive wire, and the drive portion thus controls the patch via the conductive wire. Therefore, a plurality of conductive wires are typically used between the drive portion and the patch, and during use, the conductive wires may be tangled or twisted around the body of a user. As a result, it is troublesome for the user to use and accommodate. In addition, upon accommodation, the apparatus still has a large volume.

### SUMMARY

Embodiments of the present disclosure provide a physiotherapeutic apparatus receptacle and a physiotherapeutic apparatus system to solve the technical problem in the related art.

According to one aspect of the embodiments of the present disclosure, a physiotherapeutic apparatus receptacle applicable to accommodating a physiotherapeutic apparatus is provided. The physiotherapeutic apparatus includes a first drive portion and a first patch, wherein the first patch is positioned on a side of the first drive portion and movably integrally formed with the first drive portion. The physiotherapeutic apparatus receptacle includes a housing. An accommodation chamber is defined in the housing, wherein a base is arranged at an opening of the accommodation chamber, and the base is recessed towards an interior of the accommodation chamber to define a first accommodation trough and a second accommodation trough. The first accommodation trough and the second accommodation trough are arranged adjacent to each other, and when connected to each other, the first drive portion and the first patch are simultaneously received in the first accommodation trough and the second accommodation trough respectively.

In the technical solution according to the embodiments of the present disclosure, with the base arranged in the physiotherapeutic apparatus receptacle, and the adjacently arranged accommodation troughs respectively configured to receive the drive portion and the patch of the physiotherapeutic apparatus, when connected to each other, the drive portion and the patch are simultaneously received in their corresponding accommodation troughs. Such a design facilitates conveniently receiving the physiotherapeutic apparatus. Further, the physiotherapeutic apparatus receptacle is more compact in structure, and has a relatively smaller overall thickness.

In some embodiments, the physiotherapeutic apparatus further includes a second drive portion and a second patch, wherein the second patch is positioned on a side of the second drive portion and movably integrally formed with the second drive portion. The base is recessed towards the interior of the accommodation chamber to define a third accommodation trough. The third accommodation trough is adjacently arranged on a side, far away from the first accommodation trough, of the second accommodation trough. When the first drive portion and the second drive portion are respectively received in the first accommodation trough and the third accommodation trough, the first patch and the second patch are attached to each other and both received in the second accommodation trough. When a pair of physiotherapeutic apparatuses are used, that is, two drive portions and two patches, the accommodation trough configured to receive the patches is arranged between the accommodation troughs configured to receive the drive portions such that the two patches are attached to each other and are received in the same accommodation trough. Such a design achieves a more compact internal structure of the physiotherapeutic apparatus receptacle, and the physiotherapeutic apparatus receptacle has a relatively smaller overall thickness.

In some embodiments, a first spacer is defined between the first accommodation trough and the second accommodation trough; and a second spacer is defined between the second accommodation trough and the third accommodation trough. The first spacer is configured to be inserted between the first drive portion and the first patch, and the second spacer is configured to be inserted between the second drive portion and the second patch. The spacer defined between the accommodation troughs restricts relative movement of the physiotherapeutic apparatus in the physiotherapeutic apparatus receptacle, and thus better secures the physiotherapeutic apparatus and prevents the physiotherapeutic apparatus from being damaged due to wobbling in case of displacement in the physiotherapeutic apparatus receptacle.

In some embodiments, a first printed circuit board, a battery, and a second printed circuit board are successively arranged in the accommodation chamber. The battery is electrically connected to the first printed circuit board and the second printed circuit board, the first printed circuit board is arranged corresponding to the first accommodation trough, and the second printed circuit board is arranged corresponding to the third accommodation trough. A first magnetically-attachable charge structure is arranged in the first accommodation trough, and the first printed circuit board is electrically connected to the first magnetically-attachable charge structure. A second magnetically-attachable charge structure is arranged in the third accommodation trough, and the second printed circuit board is electrically connected to the second magnetically-attachable charge structure. The part having the charging function arranged in the physiotherapeutic apparatus receptacle helps the physiotherapeutic apparatus receptacle to implement both accommodation and charging functions. In addition, the drive portions in the different accommodation troughs are respectively charged by the first printed circuit board and the second printed circuit board. In one aspect, where any of the printed circuit boards encounters a failure, charging of the other drive portion is not affected. In another aspect, the first printed circuit board and the second printed circuit board are arranged on two sides of a battery, such that the receptacle is structurally more symmetric, weight distribution of the receptacle is more even, and life time of the receptacle is greatly prolonged.

In some embodiments, the first printed circuit board or the second printed circuit board is provided with a wireless signal transmission module. The wireless signal transmission module is configured to transmit wireless signals to respectively control the first drive portion and the second drive portion of the physiotherapeutic apparatus. The wireless signal transmission module arranged in the physiotherapeutic apparatus receptacle achieves wireless remote control over the physiotherapeutic apparatus.

In some embodiments, the base is provided with an extension portion. The extension portion is extended to the interior of the accommodation chamber and configured to secure the first printed circuit board, the battery, and the second printed circuit board. The extension portion on the base helps to secure other functional parts in the accommodation chamber. In this way, where the physiotherapeutic apparatus receptacle is displaced, these functional parts in the accommodation chamber are prevented from being damaged due to wobbling.

In some embodiments, the physiotherapeutic apparatus receptacle further includes a flip cover, placed over the opening of the accommodation chamber. One side of the flip cover is rotatably connected to a side of the housing by a metal shaft, and the other side of the flip cover is magnetically attached to the housing. Connecting the flip cover to the housing by the metal shaft or magnetic attachment structure facilitates convenient closing or opening of the flip cover. In addition, the metal shaft is not prone to breakage, such that life time of the receptacle is greatly prolonged.

In some embodiments, at least one magnet is arranged on the flip cover, wherein a magnetic force sensor is arranged at a position, corresponding to the magnet, of the base. The magnetic force sensor is configured to detect opening or closing of the flip cover. With the magnet arranged on the flip cover and the magnetic force sensor arranged on the base, the magnetic force sensor determines closing or opening of the flip cover according to variations of the magnetic field, to control running of a display module.

According to another aspect of the embodiments of the present disclosure, a physiotherapeutic apparatus system is provided. The physiotherapeutic apparatus system includes the physiotherapeutic apparatus receptacle as described above, and a physiotherapeutic apparatus.

In some embodiments, the physiotherapeutic apparatus includes at least one drive portion. Each of the at least one drive portion is provided with a sound control module, and configured to receive sound control information. With the sound control module arranged on the drive portion of the physiotherapeutic apparatus, a user controls the physiotherapeutic apparatus by issuing a voice command thereto.

The above description only summarizes the technical solutions of the present disclosure. Specific embodiments of the present disclosure are described hereinafter to better and clearer understand the technical solutions of the present disclosure, to practice the technical solutions based on the disclosure of the specification and to make the above and other objectives, features and advantages of the present disclosure more apparent and understandable.

### BRIEF DESCRIPTION OF THE DRAWINGS

By reading the detailed description of preferred embodiments hereinafter, various other advantages and beneficial effects become clear and apparent for persons of ordinary skill in the art. The accompanying drawings are merely for illustrating the preferred embodiments, but shall not be construed as limiting the present disclosure. In all the accompanying drawings, like reference numerals denote like parts. In the drawings:

FIG. 1 is a perspective view of a physiotherapeutic apparatus according to some embodiments of the present disclosure;

FIG. 2 is a schematic external structural view of a physiotherapeutic apparatus receptacle according to some embodiments of the present disclosure;

FIG. 3 is a perspective view of a physiotherapeutic apparatus receptacle according to some embodiments of the present disclosure; and

FIG. 4 is a sectional view of a physiotherapeutic apparatus according to some embodiments of the present disclosure.

### DETAILED DESCRIPTION

The embodiments containing the technical solutions of the present disclosure are described in detail with reference to the accompanying drawings. The embodiments hereinafter are only used to clearly describe the technical solutions of the present disclosure. Therefore, these embodiments are only used as examples, but are not intended to limit the protection scope of the present disclosure.

Unless defined otherwise, all technical and scientific terms used herein have the same meaning as commonly understood by one of ordinary skill in the art to which the present disclosure pertains. The terms used herein in the specification of present disclosure are only intended to illustrate the specific embodiments of the present disclosure, instead of limiting the present disclosure. The terms “comprise,” “include,” and any variations thereof in the specification, claims, and the description of the drawings of the present disclosure are intended to cover a non-exclusive inclusion.

In the description of the present disclosure, the terms “first,” “second,” and the like are only used for distinguishing different objects, but shall not be understood as indication or implication of relative importance or implicit indication of the number of the specific technical features, the specific sequence or priorities. In the description of the embodiments of the present disclosure, the term “multiple” or “a plurality of” signifies at least two, unless otherwise specified.

The terms “example” and “embodiment” in this specification signify that the specific characteristic, structures or features described with reference to the embodiments may be covered in at least one embodiment of the present disclosure. This term, when appearing in various positions of the description, neither indicates the same embodiment, nor indicates an independent or optional embodiment that is exclusive of the other embodiments. A person skilled in the art would implicitly or explicitly understand that the embodiments described in this specification may be incorporated with other embodiments.

In the description of the embodiments of the present disclosure, the term “and/or” is merely an association relationship for describing associated objects, which represents that there may exist three types of relationships, for example, A and/or B may represent three situations: only A exists, both A and B exist, and only B exists. In addition, the forward-slash symbol “/” generally represents an “or” relationship between associated objects before and after the symbol.

In the description of the embodiments of the present disclosure, the term “multiple” or “a plurality of” signifies more than two (including two), unless otherwise specified. Likewise, the term “a plurality of groups” or “multiple groups” signifies more than two groups (including two groups), and the term “a plurality of pieces” or “multiple pieces” signifies more than two pieces (including two pieces).

In the description of the embodiments of the present disclosure, it should be understood that the terms “central,” “transversal,” “longitudinal,” “length,” “width,” “thickness,” “upper,” “lower,” “front,” “rear,” “left,” “right,” “vertical,” “horizontal,” “top,” “bottom,” “inner,” “outer,” “clockwise,” “counterclockwise,” “axial,” “radial,” “circumferential,” and the like indicate orientations and position relationships which are based on the illustrations in the accompanying drawings, and these terms are merely for ease and brevity of the description, instead of indicating or implying that the devices or elements shall have a particular orientation and shall be structured and operated based on the particular orientation. Accordingly, these terms shall not be construed as limiting the present disclosure.

In the description of the embodiments of the present disclosure, it should be noted that unless otherwise specified and defined, the terms “mounted,” “coupled,” “connected,” “fixed,” and derivative forms thereof shall be understood in a broad sense, which, for example, may be understood as fixed connection, detachable connection or integral connection; may be understood as mechanical connection or electrical connection, or understood as direct connection, indirect connection via an intermediate medium, or communication between the interiors of two elements or interactions between two elements. Persons of ordinary skill in the art may understand the specific meanings of the above terms in the embodiments of the present disclosure according to the actual circumstances and contexts.

A physiotherapeutic apparatus, i.e., physical therapeutic apparatus, applies physical agents to a human body for physical treatment. Corresponding to drug therapy, the physical therapy is safe and environmentally-friendly. In addition, the physical therapy has very good effects on some diseases, for example, enhancing functions of the organs of the human body or improving states of partial tissues of the human body, and the like by improving blood circulation. At present, a variety of physiotherapeutic apparatuses are available. For example, a transcutaneous electrical nerve stimulation (Tens) physiotherapeutic apparatus stimulates sensory nerves by a mild and trace current to block transmission of nerve signals of pain sensation, so as to achieve the effect of relieving pain. This process may not cause any harm to the human body. Therefore, the physiotherapeutic apparatus is welcomed by people. The physiotherapeutic apparatuses also include massage apparatuses, electric moxibustion physiotherapeutic apparatuses, and microwave therapeutic apparatuses, and the like.

The inventors of the present disclosure have noted that a physiotherapeutic apparatus generally includes a drive portion and a patch. The drive portion is connected to the patch via a conductive wire, the drive portion is configured to generate and control a stimulation current. The patch is configured to be in contact with a human body. During use, the patch usually needs to be attached to a specific part of the human body, and the stimulation current is generated by opening a switch of the drive portion, and is conducted to the patch via the conductive wire so as to generate a stimulation effect on the human body. During use, the conventional physiotherapeutic apparatus requires a conductive wire to connect the drive portion to the patch, and the conductive wire is always tangled or twisted. As a result, the physiotherapeutic apparatus is very inconvenient to use and accommodate, and has a large volume upon accommodation. In order to solve the problem of inconvenience in use and accommodation, the connection between the drive portion and the patch may be made in the form of a conductive electromagnetic attachment structure or a conductive snap or the like. This eliminates the need for conductive wires, thereby simplifying the structure of the physiotherapeutic apparatus. However, since the patch is generally large, it is difficult to accommodate the patch by first detaching the patch from the drive portion and then separately accommodating the patch. In addition, when designing a receptacle, in addition to the space for receiving the drive portion of the physiotherapeutic apparatus, there is a need to additionally define a space for receiving the patch in the receptacle. As such, accommodation is inconvenient, and an additional space is needed for receiving the patch.

In order to solve the problems of inconvenient accommodation and the need of an additional space to receive the patch, the inventors of the present disclosure have found that the structure of the physiotherapeutic apparatus receptacle may be improved, and when the physiotherapeutic apparatus receptacle is used to receive the physiotherapy apparatus, the drive portion and the patch do not need to be separated, and the drive portion and the patch may be integrally received in the receptacle. By adjacently arranging an accommodation trough configured to receive the patch and an accommodation trough configured to receive the drive portion in the receptacle, during receiving the physiotherapeutic apparatus, the drive portion and the patch do not need to be detached from each other, and during receiving the drive portion in a space for receiving the drive portion, the patch may be received in an adjacent space for receiving the patch at the same time.

In such a physiotherapeutic apparatus receptacle, spaces capable of receiving the drive portion and the patch of the physiotherapeutic apparatus are defined on the base, and no additional space for receiving the patch is needed. In this way, the structure is compact, and an overall thickness of the receptacle is relatively small. In addition, the drive portion and the patch of the physiotherapeutic apparatus may be received with no need of detaching the drive portion from the patch, such that use and receiving of the physiotherapeutic apparatus are more convenient.

The physiotherapy apparatus receptacle according to the embodiments of the present disclosure can be used, but not limitedly, for receiving a Tens physiotherapeutic apparatus, and can also be used to receive any electronic or physiotherapeutic apparatus device with a similar structure, such as a massager, an electrophysiotherapeutic apparatus, and an ultrasonic physiotherapeutic apparatus. The electrophysiotherapeutic apparatus may include a physiotherapeutic apparatus using a direct current therapy method, low frequency therapy, medium frequency therapy, high frequency therapy, or the like, for example, a direct current electrotherapeutic apparatus, a Tens physiotherapeutic apparatus, a microwave therapeutic apparatus, or the like. The ultrasonic physiotherapeutic apparatus may include an electro-ultrasonic therapeutic apparatus. With the physiotherapeutic apparatus receptacle according to the embodiments of the present disclosure, the apparatus is more conveniently received, and the receptacle has a compact structure and a relatively small overall thickness, and is convenient to carry.

Some embodiments are described with reference to a physiotherapeutic system that receives a Tens physiotherapeutic apparatus as an example.

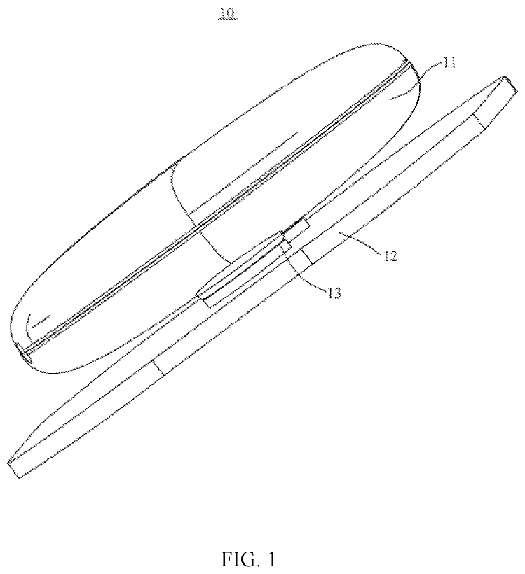

Referring to FIG. 1, a perspective view of a physiotherapeutic apparatus 10 according to some embodiments of the present disclosure is illustrated. The physiotherapeutic apparatus 10 is a Tens physiotherapeutic apparatus.

The Tens physiotherapeutic apparatus includes a drive portion 11 and a patch 12. The drive portion 11 and the patch 12 are electrically connected to each other via a connection portion 13. The drive portion 11 is a main operating device of the Tens physiotherapeutic apparatus. The drive portion 11 is provided with a battery and a printed circuit board, and is capable of generating a current with a suitable intensity and frequency and mildly transmitting a minute amount of current to the patch 12 electrically connected thereto. The patch 12 may be attached to skin of a human body, and transmit the received current to the skin, such that the current enters the human body and stimulates sensory nerves of the human body. The connection portion 13 is configured to connect the drive portion 11 and the patch 12 and is electrically conductive. The current generated by the drive portion 11 is transmitted to the patch 12 via the connection portion 13 In addition, the connection portion 13 is a detachable structure, and the user may replace the patch 12 where necessary. Optionally, the connection portion may be in a variety of forms, for example, a conductive magnetic attachment structure, a conductive snap, and the like. It should be particularly noted that in the embodiments of the present disclosure, the drive portion and the patch are integrally and movably arranged. It may be understood that during use, the drive portion and the patch are placed on the skin of the human body as an entirety.

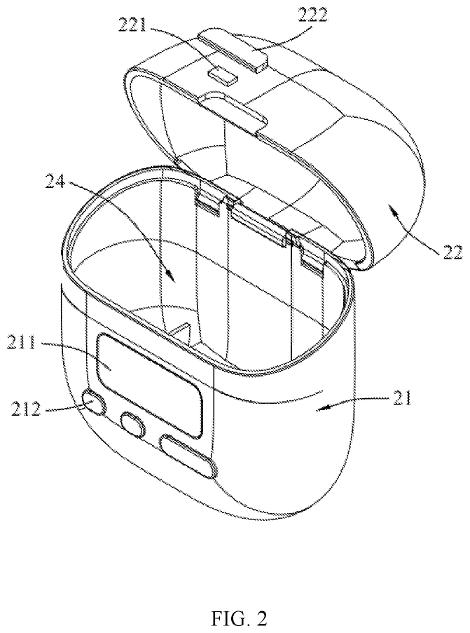

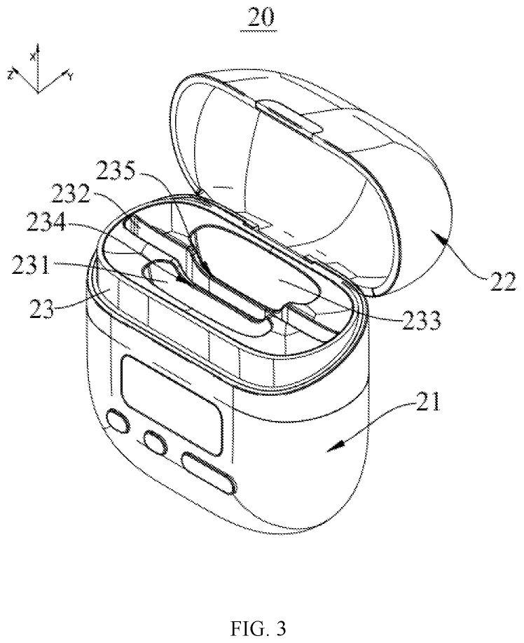

Referring to FIG. 2, a schematic external structural view of a physiotherapeutic apparatus receptacle according to some embodiments of the present disclosure is illustrated. Still referring to FIG. 3, a perspective view of a physiotherapeutic apparatus receptacle according to some embodiments of the present disclosure is illustrated. A physiotherapeutic apparatus receptacle 20 includes a housing 21, a flip cover 22, an accommodation chamber 24, and other functional parts.

The housing 21 is an assembly configured to form an accommodation chamber for receiving the other functional parts. An opening is defined in the housing 21. The opening is covered by the flip cover 22, such that an internal environment of the physiotherapeutic apparatus receptacle 20 is formed. The housing 21 may be in a variety of shapes and dimensions, for example, a rectangle, an elliptic cylinder, an irregular cylinder, or the like. Specifically, the shape and dimension of the housing 21 respectively match the shape and dimension of the physiotherapeutic apparatus 10 received therein. The housing 21 may be made of a variety of materials, for example, plastic, rubber, or the like.

The flip cover 22 refers to a part covered at the opening in the housing 21 to isolate the internal environment of the physiotherapeutic apparatus receptacle 20 from an external environment. Without any limitation, the shape and dimension of the flip cover 22 may respectively match the shape and dimension of the housing 21. Optionally, the flip cover 22 may be made of a variety of materials, for example, plastic, rubber, or the like.

The accommodation chamber 24 is a space of the internal environment, in the housing 21, for receiving the other functional parts. Besides the function of receiving the physiotherapeutic apparatus 10, the accommodation chamber 24 may also have other functions, for example, receiving a rechargeable battery, a remote control circuit, a display circuit, or the like functional parts for implementing other functions. The dimension and shape of the accommodation chamber 24 match the dimension and shape of the housing 21.

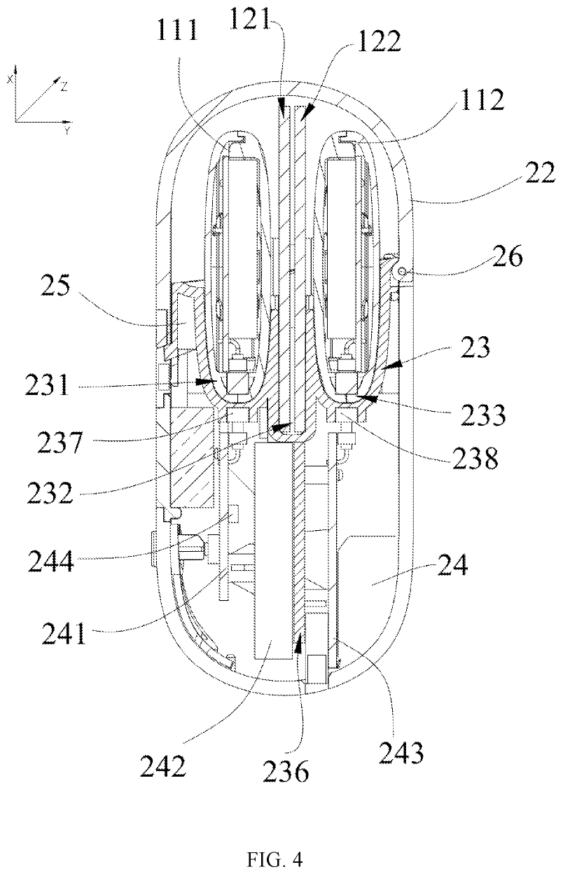

Based on the above content, to solve the problem that the conventional physiotherapeutic apparatus receptacle is inconvenient to receive the physiotherapeutic apparatus and an extra space is needed for receiving the patch, some embodiments of the present disclosure provide a new physiotherapeutic apparatus receptacle. Referring to FIG. 2 and further referring to FIG. 3 and FIG. 4, a sectional view of a physiotherapeutic apparatus system according to some embodiments of the present disclosure is illustrated.

As illustrated in FIG. 3 and FIG. 4, the present disclosure provides a physiotherapeutic apparatus receptacle 20 applicable to accommodating a physiotherapeutic apparatus 10. As illustrated in FIG. 1, the physiotherapeutic apparatus 10 includes a first drive portion 111 and a first patch 121. The first patch 121 is positioned on a side of the first drive portion 111 and movably integrally formed with the first drive portion 111. The physiotherapeutic apparatus receptacle 20 includes a housing 21. An accommodation chamber 24 is defined in the housing 21. A base 23 is arranged at an opening of the accommodation chamber 24, and the base 24 is recessed towards an interior of the accommodation chamber 24 to define a first accommodation trough 231 and a second accommodation trough 232. The first accommodation trough 231 and the second accommodation trough 232 are arranged adjacent to each other, and when connected to each other, the first drive portion 111 and the first patch 121 are simultaneously received in the first accommodation trough 231 and the second accommodation trough 232 respectively.

The base 23 is an assembly configured to receive the physiotherapeutic apparatus 10. As illustrated in FIG. 3 and FIG. 4, the base 23 is arranged at the opening of the accommodation chamber 24. That is, when the flip cover 22 is opened, the physiotherapeutic apparatus 10 is placed onto the base 23 via the opening of the accommodation chamber 24. The base 23 may be snap-fit to the housing 21 at the opening of the accommodation chamber 24, or may be arranged together with the housing 21 by adhesion or the like manners. The base 23 is recessed towards the interior of the accommodation chamber 24 to form the first accommodation trough 231 and the second accommodation trough 232. That is, in an X-axis direction on a surface of the base 23, the base 23 is recessed towards the interior of the accommodation chamber 24 to form the accommodation troughs.

The first accommodation trough 231 and the second accommodation trough 232 are arranged adjacent to each other along a Y-axis direction successively. The first accommodation trough 231 matches the structure of the drive portion 11 of the physiotherapeutic apparatus, which may be a U-shaped trough, a square-shaped trough, or a trough of any other shape. The second accommodation trough 232 matches the shape of the patch 12. In practice, generally the patch 12 has an area greater than that of the drive portion 11 but has a thickness less than that of the drive portion 11. Therefore, generally a width of the second accommodation trough 232 is greater than a width of the first accommodation trough 231, and a thickness of the second accommodation trough 232 is less than a thickness of the first accommodation trough 231. The first accommodation trough 231 may be in communication with or not in communication with the second accommodation trough 232.

As illustrated in FIG. 4, the first accommodation trough 231 is configured to receive the first drive portion 111, and the shape and dimension of the first accommodation trough 231 match the shape and dimension of the first drive portion 111. The second accommodation trough 232 is configured to receive the first patch 121. The shape and dimension of the second accommodation trough 232 match the shape and dimension of the first patch 121. It should be noted that a space for receiving one physiotherapeutic apparatus, that is, including the first drive portion 111 and the first patch 121, may be defined on the base 23; or a space for receiving a plurality of physiotherapeutic apparatuses, that is, a plurality of drive portions 11 and a plurality of patches 12, may be defined on the base 23. When the physiotherapeutic apparatus receptacle 20 is configured to receive a plurality of physiotherapeutic apparatuses 10, a plurality of first accommodation troughs 231 and a plurality of second accommodation troughs 232 may be juxtaposed in the base 23 along a Y-axis direction or a Z-axis direction to form physiotherapeutic apparatus accommodation units. A plurality of physiotherapeutic apparatuses accommodation units form a physiotherapeutic apparatus accommodation array. All the physiotherapeutic apparatus accommodation units have the same structure to receive the plurality of physiotherapeutic apparatuses 10.

During receiving the physiotherapeutic apparatus 10, a user may directly take off the entirety of the drive portion 11 and the patch 12 from the human body. Where only one drive portion and one patch are included, the drive portion and the patch do not need to be detached from each other, but are directly placed in the first accommodation trough 231 and the second accommodation trough 232. Where the user uses a plurality of physiotherapeutic apparatuses and a plurality of drive portions and a plurality of patches are included, then when taking off the plurality of physiotherapeutic apparatuses, the user does not need to detach the drive portions from the patches, but directly places the drive portion and the patch of each of the physiotherapeutic apparatuses as an entirety into a physiotherapeutic apparatus accommodation unit, and the like.

In the physiotherapeutic apparatus receptacle according to the embodiments of the present disclosure, with the first accommodation trough 231 and the second accommodation trough 232 defined on the base 23, the drive portion and the patch of the physiotherapeutic apparatus are orderly and well received, such that the physiotherapeutic apparatus receptacle 20 has a more compact internal structure and a relatively small overall thickness. Meanwhile, during receiving, the drive portion and the patch of the physiotherapeutic apparatus do not need to be dismounted or detached, but are directly placed in the receptacle, such that use and receiving of the physiotherapeutic apparatus are more convenient.

When the physiotherapeutic apparatus receptacle 20 is configured to receive a pair of physiotherapeutic apparatuses 10, that is, including two drive portions and two patches, to make the internal structure of the physiotherapeutic apparatus receptacle more compact, in some embodiments of the present disclosure, optionally, referring to FIG. 3 and FIG. 4, the physiotherapeutic apparatus 10 further includes a second drive portion 112 and a second patch 122. The second patch 122 is arranged on a side of the second drive portion 112, and is movably integrally formed with the second drive portion 112. The base 23 is recessed towards the interior of the accommodation chamber 24 to form a third accommodation trough 233. The third accommodation trough 233 is adjacently arranged on a side, away from the first accommodation trough 231, of the second accommodation trough 232. When the first drive portion 111 and the second drive portion 112 are respectively received in the first accommodation trough 231 and the third accommodation trough 233, the first patch 121 and the second patch 122 are attached to each other and both received in the second accommodation trough 232.

The third accommodation trough 233 is adjacently arranged on a side of the second accommodation trough 232, and the second accommodation trough 232 is sandwiched between the first accommodation trough 231 and the third accommodation trough 233, which are successively arranged along the Y-axis direction. The third accommodation trough 233 matches the structure of the drive portion 11 of the physiotherapeutic apparatus, which may be a U-shaped trough, a square-shaped trough, or a trough of any other shape. In practice, generally the patch 12 has an area greater than that of the drive portion 11 but has a thickness less than that of the drive portion 11. Therefore, generally a width of the third accommodation trough 233 is less than a width of the second accommodation trough 232, and a thickness of the third accommodation trough 233 is greater than a thickness of the second accommodation trough 232. The second accommodation trough 232 may be in communication with or not in communication with the third accommodation trough 233.

As illustrated in FIG. 4, the first accommodation trough 231 and the third accommodation trough 233 are respectively configured to receive the first drive portion 111 and the second drive portion 112, and the shapes and dimensions of the first accommodation trough 231 and the third accommodation trough 233 match the shapes and dimensions of the first drive portion 111 and the second drive portion 112. The second accommodation trough 232 is configured to receive the first patch 121 and the second patch 122. The shape and dimension of the second accommodation trough 232 match the shapes and dimensions of the first patch 121 and the second patch 122. It should be noted that a space for receiving a pair of physiotherapeutic apparatuses, that is, including the first drive portion 111, the first patch 121, the second drive portion 112, and the second patch 122, may be defined on the base 23; or a space for receiving a plurality of pairs of physiotherapeutic apparatuses, that is, a plurality of drive portions 11 and a plurality of patches 12, may be defined on the base 23. When the physiotherapeutic apparatus receptacle 20 is configured to receive a plurality of pair of physiotherapeutic apparatuses 10, a plurality of first accommodation troughs 231, a plurality of second accommodation troughs 232, and a plurality of third accommodation troughs 233 may be juxtaposed in the base 23 along the Y-axis direction or the Z-axis direction to form physiotherapeutic apparatus accommodation units. A plurality of physiotherapeutic apparatuses accommodation units form a physiotherapeutic apparatus accommodation array. All the physiotherapeutic apparatus accommodation units have the same structure to receive the plurality of pairs of physiotherapeutic apparatuses 10. In the embodiments of the present disclosure, description is given using only a pair of physiotherapeutic apparatuses 10 as an example.

During receiving the physiotherapeutic apparatus 10, a user may directly take off the entirety of the drive portion 11 and the patch 12 from the human body. Where only one drive portion and one patch are included, the drive portion and the patch do not need to be detached from each other, but are directly placed in the first accommodation trough 231 and the second accommodation trough 232. Where the user uses a pair of physiotherapeutic apparatuses, two drive portions and two patches are included, and then when taking off the physiotherapeutic apparatuses, the user does not need to detach the drive portions from the patches, but directly attaches the patches of the two physiotherapeutic apparatuses, respectively places the first drive portion 111 and the second drive portion 112 into the first accommodation trough 231 and the third accommodation trough 233, and simultaneously places the first patch 121 and the second patch 122 in the second accommodation trough 232, and so on.

In the physiotherapeutic apparatus receptacle according to the embodiments of the present disclosure, with the first accommodation trough 231, the second accommodation trough 232, and the third accommodation trough 233 defined on the base 23, the drive portion and the patch of the physiotherapeutic apparatus are orderly and well received, such that the physiotherapeutic apparatus receptacle 20 has a more compact internal structure and a relative small overall thickness.

For better securing of the physiotherapeutic apparatus 10 in the physiotherapeutic apparatus receptacle to prevent the physiotherapeutic apparatus 10 from being damaged due to wobbling in the physiotherapeutic apparatus receptacle 20, in some embodiments, still referring to FIG. 3, a first spacer 234 is defined between the first accommodation trough 231 and the second accommodation trough 232, and a second spacer 235 is defined between the second accommodation trough 232 and the third accommodation trough 233. The first spacer 234 is configured to be inserted between the first drive portion 111 and the first patch 121, and the second spacer 235 is configured to be inserted between the second drive portion 112 and the second patch 122.

In order that the physiotherapeutic apparatus 10 can be more conveniently taken out from the physiotherapeutic apparatus receptacle 20 and stored, generally accommodation troughs are designed to be greater than practical sizes of articles to be received therein, such that a gap is present between each of the drive portion 11 and the patch 12 and an inner wall of its corresponding accommodation trough. When two adjacent accommodation troughs are in communication with each other, the physiotherapeutic apparatus 10 has a relatively great movement space in the receptacle 20, and is thus subject to more severe wobbling in the receptacle 20. As a result, the physiotherapeutic apparatus 10 is prone to damages.

In combination with the structure of the physiotherapeutic apparatus 10 as illustrated in FIG. 1, the drive portion 11 and the patch 12 are electrically connected to each other by magnetic attachment or snap-fitting or the like. Due to presence of the connection portion 13, a slit is generally present between the drive portion 11 and the patch 12. In this case, by arranging a spacer in the physiotherapeutic apparatus receptacle 20, the movement space of the physiotherapeutic apparatus 10 in the physiotherapeutic apparatus receptacle 20 may be restricted. As illustrated in FIG. 3, a bump is arranged between the first accommodation trough 231 and the second accommodation trough 232 along a direction towards the opening of the accommodation chamber 24. The bump forms the first spacer 234, such that the first accommodation trough 231 is not in complete communication with the second accommodation trough 232. Likewise, a bump is also arranged between the second accommodation trough 232 and the third accommodation trough 233 along a direction towards the opening of the accommodation chamber 24. The bump forms the second spacer 235, such that the second accommodation trough 232 is not in complete communication with the third accommodation trough 233. The first spacer 234 needs to be inserted between the first drive portion 111 and the first patch 121, and the second spacer 235 needs to be inserted between the second drive portion 112 and the second patch 122. By filling the first spacer 234 between the first drive portion 111 and the first patch 121 and filling the second spacer 235 between the second drive portion 112 and the second patch 122, the first spacer 234 and the second spacer 235 support the physiotherapeutic apparatus 10, such that a margin for wobbling of the physiotherapeutic apparatus 10 in the physiotherapeutic apparatus receptacle 20 is restricted. A height of the first spacer 234 may be less than a depth of the first accommodation trough 231, such that the first accommodation trough 231 is in partial communication with the second accommodation trough 232; and alternatively, the height of the first spacer 234 may also be equal to or greater than the depth of the first accommodation trough 231, such that the first accommodation trough 231 is completely not in communication with the second accommodation trough 232. Likewise, a height of the second spacer 235 may be less than a depth of the third accommodation trough 233, such that the second accommodation trough 232 is in partial communication with the third accommodation trough 233; and alternatively, the height of the second spacer 235 may be equal to or greater than the depth of the third accommodation trough 233, such that the second accommodation trough 232 is completely not in communication with the third accommodation trough 233.

For matching a shape of the connection portion 13 between the drive portion 11 and the patch 12, receiving troughs (not illustrated) may be respectively defined at positions, corresponding to the connection portion 13, of the first spacer 234 and the second spacer 235, such that the connection portion 13 between the drive portion and the patch is received in the receiving troughs. In this way, wobbling of the drive portion 11 and the patch 12 is prevented.

According to the above embodiments, with the first spacer 234 defined between the first accommodation trough 231 and the second accommodation trough 232 and the second spacer 235 defined between the second accommodation trough 232 and the third accommodation trough 233, movement of the physiotherapeutic apparatus 10 in the receptacle is restricted, such that the physiotherapeutic apparatus 10 is better secured and is prevented from severe wobbling in the physiotherapeutic apparatus receptacle 20.

For a more symmetric and compact structure of the physiotherapeutic apparatus receptacle, referring to FIG. 4, in some embodiments, a first printed circuit board 241, a battery 242, and a second printed circuit board 243 are successively arranged in the accommodation chamber 24. The battery 242 is electrically connected to the first printed circuit board 241 and the second printed circuit board 243. The first printed circuit board 241 is arranged corresponding to the first accommodation trough 231. The second printed circuit board 243 is arranged corresponding to the third accommodation trough 233. A first magnetically-attachable charge structure 237 is arranged in the first accommodation trough 231, and the first printed circuit board 241 is electrically connected to the first magnetically-attachable charge structure 237. A second magnetically-attachable charge structure 238 is arranged in the third accommodation trough 233, and the second printed circuit board 243 is electrically connected to the second magnetically-attachable charge structure 238.

The physiotherapeutic apparatus 10 consumes power during operation, a capacity of a battery carried by the drive portion 11 is limited, and the battery needs to be charged upon a period of time of use. Therefore, for timely charging of the physiotherapeutic apparatus 10, the physiotherapeutic apparatus receptacle 20 may provide a charging function which is capable of charging the physiotherapeutic apparatus 10. As illustrated in FIG. 4, the battery 242 is arranged in the accommodation chamber 24. The battery 242 is a power source, which has an energy storage function and supplies power to devices. In the embodiments of the present disclosure, the battery 242 charges the first drive portion 111 by the first printed circuit board 241, and charges the second drive portion 112 by the second printed circuit board 243. With the first printed circuit board 241 and the second printed circuit board 243, the two drive portions are respectively charged. With such configurations, in one aspect, where any of the printed circuit boards encounters a failure, charging of the other drive portion is not affected; and in another aspect, the first printed circuit board 241 and the second printed circuit board 243 are symmetrically arranged on two sides of a battery, such that the receptacle is structurally more symmetric, weight distribution of the receptacle is more even, and life time of the receptacle is greatly prolonged. The shape and dimension of the battery 242 match the space formed by the accommodation chamber 24 and the base 23. The shape of the battery 242 may be a rectangle, a square, a cylinder, or the like. In addition, a capacity of the battery 242 is far greater than the capacity of the battery carried by the drive portion of the physiotherapeutic apparatus.

The first printed circuit board 241 and the second printed circuit board 243 are respectively configured to charge the drive portions received in the first accommodation trough 231 and the third accommodation trough 233. As illustrated in FIG. 4, the first printed circuit board 241 is arranged corresponding to the first accommodation trough 231 and is electrically connected to the first magnetically-attachable charge structurev237 in the first accommodation trough 231; and likewise, the second printed circuit board 243 is arranged corresponding to the third accommodation trough 233 and is electrically connected to the second magnetically-attachable charge structure 238 in the third accommodation trough 233. The dimension of the first printed circuit board 241 matches a distance between the first accommodation trough 231 and the housing 21, that is, matching a distance between a bottom of the first accommodation trough 231 and the housing 21 along an X-axis direction. Likewise, the dimension of the second printed circuit board 243 matches a distance between the third accommodation trough 233 and the housing 21, that is, matching a distance between a bottom of the third accommodation trough 233 and the housing 21 along the X-axis direction. Optionally, each of the first magnetically-attachable charge structure 237and the second magnetically-attachable charge structure 238 includes a spring interfacing pin.

As illustrated in FIG. 4, the first printed circuit board 241, the battery 242, and the second printed circuit board 243 are successively vertically arranged in the accommodation chamber 24, with the successive arrangement along a Y-axis direction. The vertical arrangement makes the physiotherapeutic apparatus receptacle 20 flat, and thus achieves a compact and simple internal structure therefor, such that the physiotherapeutic apparatus receptacle 20 has a relatively smaller thickness.

When the first drive portion 111 and the second drive portion 112 are respectively received in the first accommodation trough 231 and the third accommodation trough 233, a current flows from the battery 242, flows through the first circuit board 241 and the second printed circuit board 243 to the first accommodation trough 231 and the third accommodation trough 233, and finally charges the first drive portion 111 and the second drive portion 112 by the first magnetically-attachable charge structure 237 and the second magnetically-attachable charge structure 238.

In practice, generally an operating state of the physiotherapeutic apparatus 10 is adjustable. Therefore, the drive portion 11 of the physiotherapeutic apparatus 10 is provided with a control key. The control key is configured to control the operating state of the physiotherapeutic apparatus 10. However, when the physiotherapeutic apparatus 10 is placed on the back of a user, it is hard for the user to touch and thus operate the physiotherapeutic apparatus 10 on his or her own strength. In addition, in order to allow the user to check state information of the physiotherapeutic apparatus 10, generally a display or an indicator may be arranged on the drive portion 11. The display is configured to display the state information of the physiotherapeutic apparatus 10. The indicator indicates different state information of the physiotherapeutic apparatus 10 by emitting lights of different colors or different luminances. However, in one aspect, when the physiotherapeutic apparatus 10 is placed on the back, the shoulder or the part where the state information is unreachable, the user fails to acquire the state information of the physiotherapeutic apparatus 10 by the display or the indicator on the drive portion 11. In another aspect, the information indicated by the indicator is limited and fuzzy, and the user fails to accurately acquire the state information of the physiotherapeutic apparatus 10.

To address the above problem, in some embodiments, as illustrated in FIG. 2 and FIG. 4, a display module 211 and a key 212 are arranged on a side of the housing 21. The display module 211 and the key 212 are respectively electrically connected to the first printed circuit board 241 and the second printed circuit board 243. The display module 211 is configured to display state information of the first drive portion 111 and the second drive portion 112. The key 212 is configured to control the first drive portion 111 and the second drive portion 112.

The display module 211 includes a display screen and an information receiver. The information receiver is configured to receive state information of the drive portion. The state information may include, but is not limited to, an operating mode, a power balance, a current intensity, and the like of the drive portion. The display screen is configured to display the information received by the information receiver. The key 212 is configured to control the drive portion, for example, switching the operating mode, regulating the current frequency, regulating the current intensity, and the like. The display module 211 and the key 212 are respectively electrically connected to the first printed circuit board 241 and the second printed circuit board 243, and transfer information by the first printed circuit board 241 and the second printed circuit board 243.

With the display module 211 and the key 212 arranged on the side of the housing 21, in one aspect, regardless of on which part of the human body the physiotherapeutic apparatus 10 is placed, the physiotherapeutic apparatus 10 is conveniently operated; and in another aspect, the state information of the physiotherapeutic apparatus 10 is conveniently checked by the physiotherapeutic apparatus receptacle 20, and the display module 211 is capable of displaying in more detail the state information of the physiotherapeutic apparatus 10.

In practice, for controlling the physiotherapeutic apparatus 10 by the physiotherapeutic apparatus receptacle 20, corresponding communication modules need to be arranged on the physiotherapeutic apparatus receptacle 20 and the physiotherapeutic apparatus 10. In case of wired communication, the physiotherapeutic apparatus receptacle 20 and the physiotherapeutic apparatus 10 need to be connected by a conductive wire. However, during use, the conductive wire is prone to twist or tangle, which thus restricts reachable distances of the physiotherapeutic apparatus receptacle 20 and the physiotherapeutic apparatus 10. Therefore, in some embodiments, the physiotherapeutic apparatus 10 is controlled by the physiotherapeutic apparatus receptacle 20 in a wireless fashion. Specifically, a wireless signal transmission module244 is arranged on the first printed circuit board 241 or the second printed circuit board 243. The wireless signal transmission module 244 is configured to transmit wireless signals to respectively control the first drive portion 111 and the second drive portion 112.

The wireless signal transmission module 244 may transmit various wireless signals according to control information of the key to control the physiotherapeutic apparatus 10. The wireless signals include, but are not limited to, a mode switch signal, a current enhance signal, a current decay signal, and the like. The wireless communication may be implemented by a variety of fashions, for example, Bluetooth communication, Wi-Fi communication, and the like.

According to the above embodiments, the physiotherapeutic apparatus receptacle 20 controls the physiotherapeutic apparatus 10 based on wireless communication. During use, in one aspect, the distance between the physiotherapeutic apparatus 10 and the physiotherapeutic apparatus receptacle 20 is not limited; and in another aspect, twists or tangles of the conductive wire are prevented, such that the use of the physiotherapeutic apparatus 10 is more convenient.

For better securing of other functional parts in the accommodation chamber 24 and for prevention of damages caused due to wobbling of the other functional parts in the accommodation chamber 24, in some embodiments, still referring to FIG. 4, the base 23 is provided with an extension portion 236. The extension portion 236 is extended to the interior of the accommodation chamber 24, and is configured to secure the first printed circuit board 241, the battery 242, and the second printed circuit board 243.

Where no corresponding securing part is arranged in the accommodation chamber 24, when the physiotherapeutic apparatus receptacle 20 is displaced, the other functional parts in the accommodation chamber 24 are wobble, thereby causing damages to the other functional parts. Therefore, securing parts need to be arranged in the accommodation chamber 24 to secure the other functional parts. As illustrated in FIG. 4, the extension portion 236 is arranged on the base 23, and may be in a variety of shapes, for example, a columnar shape, a plate shape, or the like. The extension portion 236 is vertically arranged in the accommodation chamber 24. That is, the bottom of the base 23 is extended, along the X-axis direction, towards the interior of the accommodation chamber 24 by a specific length to form the extension portion 236. Optionally, the extension portion 236 and the base 23 may be integrally formed, or the extension portion 236 may be arranged as a separate part on the base 23.

According to the above embodiments, with the extension portion 236 on the base 23, in one aspect, the other functional parts in the accommodation chamber 24 are supported and secured, such that these functional parts are prevented from wobbling; and in another aspect, with the vertical arrangement of the extension portion 236 in the accommodation chamber 24, the physiotherapeutic apparatus receptacle has a more compact internal structure.

In some embodiments, still referring to FIG. 2, the physiotherapeutic apparatus receptacle 20 further includes a flip cover 22. The flip cover 22 is covered at the opening of the accommodation chamber 24. One side of the flip cover 22 is rotatably connected to a side of the housing 21 by a metal shaft 26 (as shown in FIG. 4), and the other side of the flip cover 22 is magnetically attached to the housing 21.

In the physiotherapeutic apparatus receptacle 20, an internal environment isolated from an external environment typically needs to be formed by cooperation between the flip cover 22 and the housing 21. Generally, one sides of the flip cover 22 and the housing 21 are connected by a plastic rotation shaft. However, the plastic rotation shaft is prone to breakage, and as a result, life time of the physiotherapeutic apparatus receptacle 20 is affected. In the present disclosure, one side of the flip cover 22 is rotatably connected to a side of the housing 21 by a metal shaft 26, and the other side of the flip cover 22 is magnetically attached to the housing 21. The metal shaft 26 is made of a variety of materials, for example, non-stainless steel, iron, aluminum alloy, or the like. In addition, when the flip cover 22 is closed, the user fails to view, from the exterior, the interior of the physiotherapeutic apparatus receptacle 20. Therefore, the flip cover 22 may be made of a transparent or semi-transparent material.

According to the above embodiments, with one sides of the flip cover 22 and the housing 21 connected magnetically and the other sides rotatably connected by the metal shaft 26, closing or opening of the flip cover 22 with respect to the housing 21 is more convenient, and the metal shaft 26 is not prone to breakage, such that life time of the receptacle is greatly prolonged. In addition, with the flip cover 22 made of the transparent or semi-transparent material, when the flip cover 22 and the housing 21 are in a closed state, the user may directly check details about the physiotherapeutic apparatus 10 in the receptacle through the flip cover.

In some embodiments, still referring to FIG. 2 and FIG. 4, at least one magnet 221 is arranged on the flip cover 22. A magnetic force sensor 25 is arranged at a position, corresponding to the magnet 221, of the base 23. The magnetic force sensor 25 is configured to detect opening or closing of the flip cover 22.

The magnet 221, in addition to acting as a part for magnetically connecting the flip cover 22 and the housing 21, may also cooperate with the magnetic force sensor 25 to detect opening or closing of the flip cover 22. The magnet 221 generates a magnetic field, and magnetic field information at different positions is related to relative distances, directions and the like between the positions and the magnet 221. For example, in the same direction, magnetic field information at points whose distances from the magnet 221 is different; and in different directions, magnetic field information at points whose distances from the magnet 221 is also different. The magnetic field information may be, but is not limited to, a direction of the magnetic field, a magnitude of a magnetic field force, or the like.

The magnetic force sensor 25 is configured to detect a magnetic field around the magnetic force sensor 25. A controller acquires related magnetic field information, and determines the state of the flip cover according to the magnetic field information to control operation of the display module 211. When the magnet 221 is displaced with the flip cover, a relative direction and direction between the magnet 221 and the magnetic force sensor 25 are correspondingly changed, and the magnetic field information detected by the magnetic force sensor 25 is also changed. Therefore, the magnetic force sensor 25 is capable of detecting opening or closing of the flip cover 22 according to the detected magnetic field information. In addition, when the flip cover 22 is made of a transparent or semi-transparent material, where the magnet 221 is arranged on the flip cover 22, appearance of the receptacle may be affected. Therefore, for beautiful appearance of the receptacle 20, a partially-opaque structure 222 may be arranged on the flip cover 22. The opaque structure 222 may be, but is not limited to, an opaque logo structure, a trim structure, or the like, and the magnet 221 is embedded in the opaque structure 222. Further, the opaque structure 222 has an overall thickness not exceeding 2 mm.

When the flip cover 22 is opened, the display module 211 may immediately operate, related information of the physiotherapeutic apparatus 10 may be displayed on the display screen of the display module 211. The related information may be, but is not limited to, an operating mode, an output current intensity, a power balance and the like of the physiotherapeutic apparatus 10. In this way, the user may adjust operating state information of the physiotherapeutic apparatus 10 before use.

Some embodiments of the present disclosure further provide a physiotherapeutic apparatus system. The system includes the physiotherapeutic apparatus receptacle 20 and the physiotherapeutic apparatus 10 as described in any of the above embodiments. The physiotherapeutic apparatus 10 is received in the physiotherapeutic apparatus receptacle 20.

In some embodiments, for better control over the physiotherapeutic apparatus, the drive portion of the physiotherapeutic apparatus 10 is provided with a sound control module. The sound control module is configured to receive sound control information. Specifically, the physiotherapeutic apparatus 10 may be wirelessly and remotely controlled by sound control.

It should be finally noted that the above-described embodiments are merely for illustration of the present disclosure, but are not intended to limit the present disclosure. Although the present disclosure is described in detail with reference to these embodiments, a person skilled in the art may also make various modifications to the technical solutions disclosed in the embodiments, or make equivalent replacements to a part of or all technical features contained therein. Such modifications or replacement, made without departing from the principles of the present disclosure, shall fall within the scope defined by the claims and the specification of the present disclosure. Especially, various technical features mentioned in various embodiments may be combined in any fashion as long as there is no structural conflict. The present disclosure is not limited to the specific embodiments described herein in this specification, but also includes all the technical solutions falling within the scope subject to the appended claims.

What is claimed is:

1. A physiotherapeutic apparatus receptacle, applicable to accommodating a physiotherapeutic apparatus, the physiotherapeutic apparatus comprising a first drive portion and a first patch, the first patch being positioned on a side of the first drive portion and movably integrally formed with the first drive portion;
the physiotherapeutic apparatus receptacle comprising a housing;
wherein an accommodation chamber is defined in the housing, a base being arranged at an opening of the accommodation chamber, the base being recessed towards an interior of the accommodation chamber to define a first accommodation trough and a second accommodation trough;
wherein the first accommodation trough and the second accommodation trough are arranged adjacent to each other, and when connected to each other, the first drive portion and the first patch are simultaneously received in the first accommodation trough and the second accommodation trough respectively.

2. The physiotherapeutic apparatus receptacle according to claim 1, wherein the shape and dimension of the first accommodation trough match the shape and dimension of the first drive portion, the shape and dimension of the second accommodation trough match the shape and dimension of the first patch; and wherein a width of the second accommodation trough is greater than a width of the first accommodation trough, and a thickness of the second accommodation trough is less than a thickness of the first accommodation trough.

3. The physiotherapeutic apparatus receptacle according to claim 1, wherein the physiotherapeutic apparatus further comprises a second drive portion and a second patch, the second patch being positioned on a side of the second drive portion and movably integrally formed with the second drive portion; and
the base is recessed towards the interior of the accommodation chamber to define a third accommodation trough;
wherein the third accommodation trough is adjacently arranged on a side, far away from the first accommodation trough, of the second accommodation trough; and
wherein when the first drive portion and the second drive portion are respectively received in the first accommodation trough and the third accommodation trough, the first patch and the second patch are attached to each other and both received in the second accommodation trough.

4. The physiotherapeutic apparatus receptacle according to claim 3, wherein the shape and dimension of the third accommodation trough match the shape and dimension of the second drive portion, a width of the third accommodation trough is less than a width of the second accommodation trough, and a thickness of the third accommodation trough is greater than a thickness of the second accommodation trough.

5. The physiotherapeutic apparatus receptacle according to claim 3, wherein
a first spacer is defined between the first accommodation trough and the second accommodation trough; and
a second spacer is defined between the second accommodation trough and the third accommodation trough;
wherein the first spacer is configured to be inserted between the first drive portion and the first patch, and the second spacer is configured to be inserted between the second drive portion and the second patch.

6. The physiotherapeutic apparatus receptacle according to claim 5, wherein a first printed circuit board, a battery, and a second printed circuit board are successively arranged in the accommodation chamber;
wherein the battery is electrically connected to the first printed circuit board and the second printed circuit board, the first printed circuit board is arranged corresponding to the first accommodation trough, and the second printed circuit board is arranged corresponding to the third accommodation trough;
wherein a first magnetically-attachable charge structure is arranged in the first accommodation trough, and the first printed circuit board is electrically connected to the first magnetically-attachable charge structure; and
wherein a second magnetically-attachable charge structure is arranged in the third accommodation trough, and the second printed circuit board is electrically connected to the second magnetically-attachable charge structure.

7. The physiotherapeutic apparatus receptacle according to claim 6, wherein the first printed circuit board and the second printed circuit board are symmetrically arranged on two sides of a battery.

8. The physiotherapeutic apparatus receptacle according to claim 6, wherein a dimension of the first printed circuit board matches a distance between the first accommodation trough and the housing, a dimension of the second printed circuit board matches a distance between the third accommodation trough and the housing.

9. The physiotherapeutic apparatus receptacle according to claim 6, wherein the first printed circuit board, the battery, and the second printed circuit board are successively vertically arranged in the accommodation chamber.

10. The physiotherapeutic apparatus receptacle according to claim 6, wherein a display module and a key are arranged on a side of the housing, the display module and the key being respectively electrically connected to the first printed circuit board and the second printed circuit board, the display module being configured to display state information of the first drive portion and the second drive portion, the key being configured to control the first drive portion and the second drive portion.

11. The physiotherapeutic apparatus receptacle according to claim 6, wherein the first printed circuit board or the second printed circuit board is provided with a wireless signal transmission module, the wireless signal transmission module being configured to transmit wireless signals to respectively control the first drive portion and the second drive portion of the physiotherapeutic apparatus.

12. The physiotherapeutic apparatus receptacle according to claim 6, wherein the base is provided with an extension portion, the extension portion being extended to the interior of the accommodation chamber and configured to secure the first printed circuit board, the battery, and the second printed circuit board.

13. The physiotherapeutic apparatus receptacle according to claim 12, wherein the extension portion is arranged on the base, and the shape of the extension portion is a columnar shape or a plate shape, and wherein the extension portion is vertically arranged in the accommodation chamber.

14. The physiotherapeutic apparatus receptacle according to claim 1, further comprising: a flip cover, placed over the opening of the accommodation chamber;
wherein one side of the flip cover is rotatably connected to a side of the housing by a metal shaft, and the other side of the flip cover is magnetically attached to the housing.

15. The physiotherapeutic apparatus receptacle according to claim 14, wherein at least one magnet is arranged on the flip cover, a magnetic force sensor being arranged at a position, corresponding to the magnet, of the base;
wherein the magnetic force sensor is configured to detect opening or closing of the flip cover.

16. The physiotherapeutic apparatus receptacle according to claim 15, wherein the display module is configured to operate when the flip cover is opened, and related information of the physiotherapeutic apparatus being displayed on a display screen of the display module, wherein the related information comprises one or more of an operating mode, an output current intensity and a power balance of the physiotherapeutic apparatus.

17. A physiotherapeutic apparatus system, comprising: the physiotherapeutic apparatus receptacle and the physiotherapeutic apparatus as defined in claim 1;
wherein the physiotherapeutic apparatus is received in the physiotherapeutic apparatus receptacle.

18. The physiotherapeutic apparatus system according to claim 17, wherein the physiotherapeutic apparatus comprises at least one drive portion, each of the at least one drive portion being provided with a sound control module, and configured to receive sound control information.
